SEQUENCE LISTING (69 sequences)

SEQ ID NO: 1 - protein, 8 aa, Artificial Sequence
  CDR1-H famille 1
GYTFTDYS

SEQ ID NO: 2 - protein, 8 aa, Artificial Sequence
  CDR2-H famille 1
  misc_feature (3)..(3): Xaa can be any naturally occurring amino acid
  misc_feature (8)..(8): Xaa can be any naturally occurring amino acid
ISXYYGDX

SEQ ID NO: 3 - protein, 11 aa, Artificial Sequence
  CDR3-H famille 1
  misc_feature (4)..(6): Xaa can be any naturally occurring amino acid
  misc_feature (8)..(10): Xaa can be any naturally occurring amino acid
ARNXXXYXXXY

SEQ ID NO: 4 - protein, 6 aa, Artificial Sequence
  CDR1-L famille 1
  misc_feature (4)..(4): Xaa can be any naturally occurring amino acid
QDIXNY

SEQ ID NO: 5 - skipped

SEQ ID NO: 6 - protein, 9 aa, Artificial Sequence
  CDR3-L famille 1
  misc_feature (4)..(4): Xaa can be any naturally occurring amino acid
QQGXTLPWT

SEQ ID NO: 7 - protein, 8 aa, Artificial Sequence
  CDR1-H famille 2
  misc_feature (7)..(7): Xaa can be any naturally occurring amino acid
GYTFTDXS

SEQ ID NO: 8 - protein, 8 aa, Artificial Sequence
  CDR2-H famille 2
  misc_feature (7)..(7): Xaa can be any naturally occurring amino acid
INTETGXP

SEQ ID NO: 9 - protein, 14 aa, Artificial Sequence
  CDR3-H famille 2
  misc_feature (1)..(1): Xaa can be any naturally occurring amino acid
  misc_feature (12)..(12): Xaa can be any naturally occurring amino acid
XRNGYYVGYYAXDY

SEQ ID NO: 10 - protein, 5 aa, Artificial Sequence
  CDR1-L famille 2
  misc_feature (4)..(4): Xaa can be any naturally occurring amino acid
SSVXY

SEQ ID NO: 11 - skipped

SEQ ID NO: 12 - protein, 9 aa, Artificial Sequence
  CDR3-L famille 2
  misc_feature (8)..(8): Xaa can be any naturally occurring amino acid
QQRRSYPXT

SEQ ID NO: 13 - protein, 8 aa, Artificial Sequence
  CDR1-H-IMGT-122A2
GYTFTDYS

SEQ ID NO: 14 - protein, 8 aa, Artificial Sequence
  CDR2-H-IMGT-122A2
ISTYYGDS

SEQ ID NO: 15 - protein, 11 aa, Artificial Sequence
  CDR3-H-IMGT-122A2
ARNGNFYVMDY

SEQ ID NO: 16 - protein, 6 aa, Artificial Sequence
  CDR1-L-IMGT-122A2
QDISNY

SEQ ID NO: 17 - skipped

SEQ ID NO: 18 - protein, 9 aa, Artificial Sequence
  CDR3-L-IMGT-122A2
QQGNTLPWT

SEQ ID NO: 19 - protein, 8 aa, Artificial Sequence
  CDR1-H-IMGT-102E9
GYTFTDYS

SEQ ID NO: 20 - protein, 8 aa, Artificial Sequence
  CDR2-H-IMGT-102E9
INTETGEP

SEQ ID NO: 21 - protein, 14 aa, Artificial Sequence
  CDR3-H-IMGT-102E9
TRNGYYVGYYAMDY

SEQ ID NO: 22 - protein, 5 aa, Artificial Sequence
  CDR1-L-IMGT-102E9
SSVIY

SEQ ID NO: 23 - skipped

SEQ ID NO: 24 - protein, 9 aa, Artificial Sequence
  CDR3-L-IMGT-102E9
QQRRSYPFT

SEQ ID NO: 25 - protein, 8 aa, Artificial Sequence
  CDR1-H-IMGT-104C12
GYTFTDYS

SEQ ID NO: 26 - protein, 8 aa, Artificial Sequence
  CDR2-H-IMGT-104C12
ISPYYGDT

SEQ ID NO: 27 - protein, 11 aa, Artificial Sequence
  CDR3-H-IMGT-104C12
ARNDDYYRFAY

SEQ ID NO: 28 - protein, 6 aa, Artificial Sequence
  CDR1-L-IMGT-104C12
QDINNY

SEQ ID NO: 29 - skipped

SEQ ID NO: 30 - protein, 9 aa, Artificial Sequence
  CDR3-L-IMGT-104C12
QQGKTLPWT

SEQ ID NO: 31 - protein, 8 aa, Artificial Sequence
  CDR1-H-IMGT-114D11
GYTFTDSS

SEQ ID NO: 32 - protein, 8 aa, Artificial Sequence
  CDR2-H-IMGT-114D11
INTETGGP

SEQ ID NO: 33 - protein, 14 aa, Artificial Sequence
  CDR3-H-IMGT-114D11
ARNGYYVGYYALDY

SEQ ID NO: 34 - protein, 5 aa, Artificial Sequence
  CDR1-L-IMGT-114D11
SSVFY

SEQ ID NO: 35 - skipped

SEQ ID NO: 36 - protein, 9 aa, Artificial Sequence
  CDR3-L-IMGT-114D11
QQRRSYPYT

SEQ ID NO: 37 - protein, 8 aa, Artificial Sequence
  CDR1-H-IMGT-104E10
GYTFTDYS

SEQ ID NO: 38 - protein, 8 aa, Artificial Sequence
  CDR2-H-IMGT-104E10
INTETGEP

SEQ ID NO: 39 - protein, 14 aa, Artificial Sequence
  CDR3-H-IMGT-104E10
ARNGYYVGYYAMDY

SEQ ID NO: 40 - protein, 5 aa, Artificial Sequence
  CDR1-L-IMGT-104E10
SSVIY

SEQ ID NO: 41 - skipped

SEQ ID NO: 42 - protein, 9 aa, Artificial Sequence
  CDR3-L-IMGT-104E10
QQRRSYPYT

SEQ ID NO: 43 - protein, 118 aa, Artificial Sequence
  VH-122A2
QVQLQQSGAELVRPGVSVKISCKGSGYTFTDYSMHWVKQSHAKSLEWIGVISTYYGDSNYNQKFKGKATMTVDKSSTTAYMELARLTSEDSAIYYCARNGNFYVMDYWGQGTSVTVSS

SEQ ID NO: 44 - protein, 121 aa, Artificial Sequence
  VH-102E9
QIHLVQSGPDLKKPGETVKISCKASGYTFTDYSMHWVKQAPGKGLKWMGWINTETGEPTYADDFKGRFAFSLESSASTAFLQINNLKNEDTSTYFCTRNGYYVGYYAMDYWGQGTSVTVSS

SEQ ID NO: 45 - protein, 118 aa, Artificial Sequence
  VH-104C12
QVQLQQSGAELVGPGVSVKISCKGSGYTFTDYSMHWVKQSHAKSLEWIGVISPYYGDTNYNQKFKGKATMTVDKSSSTAYMELASLTSEDSAIYFCARNDDYYRFAYWGQGTLVTVSA

SEQ ID NO: 46 - protein, 121 aa, Artificial Sequence
  VH-114D11
QIQLVQSGPELKKPGETVKISCKASGYTFTDSSMHWVQQAPNKGLKWMGWINTETGGPTYADDFKGRFAFSLETSARTAYLQINNLKNEDTATYFCARNGYYVGYYALDYWGQGTSVTVSS

SEQ ID NO: 47 - protein, 121 aa, Artificial Sequence
  VH-104E10
QIQLVQSGPELKKPGETVKISCKASGYTFTDYSMHWVKQAPGKGLKWMGWINTETGEPTYADDFKGRFAFSLETSATTAYLQINNFKNEDTATYFCARNGYYVGYYAMDYWGQGTSVTVSS

SEQ ID NO: 48 - protein, 107 aa, Artificial Sequence
  VL-122A2
DIQMTQTTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIK

SEQ ID NO: 49 - protein, 106 aa, Artificial Sequence
  VL-102E9
QIVLTQSPAIMSASPGEKVTITCSASSSVIYIHWFQQKPGTSPKLWIYSTSYLASGVPARFSGSGSGTSYSLTISRMEAEDAATYYCQQRRSYPFTFGGGTKLEIK

SEQ ID NO: 50 - protein, 107 aa, Artificial Sequence
  VL-104C12
DLQMTQTPSSLSASLGDRVTISCRASQDINNYLSWYQEKPDGTFKLLIYYTSRLHSGVPSRFSGSGSGTDYSLTVRNLEQEDIGTYFCQQGKTLPWTFGGGTKLEIR

SEQ ID NO: 51 - protein, 106 aa, Artificial Sequence
  VL-114D11
QIVLTQSPAIMSASPGEKVTITCSASSSVFYMHWFQQKPGTSPKLWIYSTSNLASGVPARFSGSGSGTSYSLTISRMEAEDAATYYCQQRRSYPYTFGGGTKLEIK

SEQ ID NO: 52 - protein, 106 aa, Artificial Sequence
  VL-104E10
QIVLTQSPAIMSASPGEKVTMTCSASSSVIYMHWFQQKPGTSPKLWIYSTSNLASGVPARFSGSGSGTSYSLTISRMEAEDAATYYCQQRRSYPYTFGGGTKLEIK

SEQ ID NO: 53 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 107 aa, Homo sapiens [sequence omitted]

SEQ ID NO: 55 - protein, 447 aa, Artificial Sequence
  VH-CH-122A2
QVQLQQSGAELVRPGVSVKISCKGSGYTFTDYSMHWVKQSHAKSLEWIGVISTYYGDSNYNQKFKGKATMTVDKSSTTAYMELARLTSEDSAIYYCARNGNFYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 450 aa, Artificial Sequence
  VH-CH-102E9
QIHLVQSGPDLKKPGETVKISCKASGYTFTDYSMHWVKQAPGKGLKWMGWINTETGEPTYADDFKGRFAFSLESSASTAFLQINNLKNEDTSTYFCTRNGYYVGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 447 aa, Artificial Sequence
  VH-CH-104C12
QVQLQQSGAELVGPGVSVKISCKGSGYTFTDYSMHWVKQSHAKSLEWIGVISPYYGDTNYNQKFKGKATMTVDKSSSTAYMELASLTSEDSAIYFCARNDDYYRFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 450 aa, Artificial Sequence
  VH-CH-114D11
QIQLVQSGPELKKPGETVKISCKASGYTFTDSSMHWVQQAPNKGLKWMGWINTETGGPTYADDFKGRFAFSLETSARTAYLQINNLKNEDTATYFCARNGYYVGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 450 aa, Artificial Sequence
  VH-CH-104E10
QIQLVQSGPELKKPGETVKISCKASGYTFTDYSMHWVKQAPGKGLKWMGWINTETGEPTYADDFKGRFAFSLETSATTAYLQINNFKNEDTATYFCARNGYYVGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 214 aa, Artificial Sequence
  VL-CL-122A2
DIQMTQTTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 213 aa, Artificial Sequence
  VL-CL-102E9
QIVLTQSPAIMSASPGEKVTITCSASSSVIYIHWFQQKPGTSPKLWIYSTSYLASGVPARFSGSGSGTSYSLTISRMEAEDAATYYCQQRRSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 214 aa, Artificial Sequence
  VL-CL-104C12
DLQMTQTPSSLSASLGDRVTISCRASQDINNYLSWYQEKPDGTFKLLIYYTSRLHSGVPSRFSGSGSGTDYSLTVRNLEQEDIGTYFCQQGKTLPWTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 213 aa, Artificial Sequence
  VL-CL-114D11
QIVLTQSPAIMSASPGEKVTITCSASSSVFYMHWFQQKPGTSPKLWIYSTSNLASGVPARFSGSGSGTSYSLTISRMEAEDAATYYCQQRRSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 213 aa, Artificial Sequence
  VL-CL-104E10
QIVLTQSPAIMSASPGEKVTMTCSASSSVIYMHWFQQKPGTSPKLWIYSTSNLASGVPARFSGSGSGTSYSLTISRMEAEDAATYYCQQRRSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 468 aa, Artificial Sequence
  channe lourde de trastuzumab
MEFGLSWLFLVAILKGVQCEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 236 aa, Artificial Sequence
  channe lighre de trastuzumab
MDMRVPAQLLGLLLLWLRGARCDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 1413 bp, Artificial Sequence
  siquence nucliique codant channe lourde de trastuzumab
atggagttcggcctgagctggctgttcctggtggccatcctgaagggcgtgcagtgcgaggtgcagctggtcgagagcggaggaggactggtccagcctggcggcagcctgagactgagctgcgccgccagcggcttcaacatcaaggacacctacatccactgggtgcgccaggctccagggaaagggctcgaatgggtggccaggatctaccccaccaacggctacaccagatacgccgacagcgtgaagggcaggttcaccatcagcgccgacaccagcaagaacaccgcctacctgcagatgaacagcctgagggccgaggacaccgccgtgtactactgcagcagatggggtggggatggcttctacgccatggactactgggggcagggcacactggtcacagtctccagcgccagcaccaagggccccagcgtgttccccctggctccttcctctaaatccacaagcggcggcaccgctgccctgggctgcctggtgaaggactacttccccgagcccgtgaccgtgtcttggaactctggcgccctgacctccggcgtgcacaccttccccgccgtgctgcagagcagcggcctgtacagcctgagcagcgtggtgaccgtgccctcttcctctctcggaacacagacctacatctgcaacgtgaaccacaagcccagcaacaccaaggtggacaagaaggtggagcccaagagctgcgacaagacccatacatgtcctccctgtcctgctcctgagctgctgggcggaccctccgtgttcctgttcccccccaagcccaaggacaccctgatgatcagcaggacccccgaggtgacctgcgtggtggtggacgtgtcccacgaggaccctgaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagcccccatcgaaaagaccatcagcaaggccaagggccagcctcgcgagccccaggtgtacaccctgcccccctcccgcgacgagctgaccaagaaccaggtgtccctgacctgtctggtgaagggcttctaccccagcgatatcgccgtggagtgggagagcaacggccagcccgagaacaactacaagaccaccccccctgtgctggacagcgacggcagcttcttcctgtacagcaagctgaccgtggacaagagcaggtggcagcagggaaatgtcttttcctgttccgtcatgcatgaagctctgcacaaccactacacccagaagtccctgagcctgagccccggcaagtgatag

SEQ ID NO: 68 - DNA, 711 bp, Artificial Sequence
  siquence nucliique codant channe lighre de trastuzumab
atggacatgagagtgcctgcccagctcctgggactcctcctcctgtggctcaggggtgctcgctgcgatatccagatgactcagtctccttcttccctctccgccagcgtgggcgacagagtgaccatcacctgcagggccagccaggacgtgaacaccgccgtggcctggtatcagcagaagcccggcaaggcccccaagctgctgatctacagcgccagcttcctgtacagcggcgtgcccagcaggttcagcggcagcagaagcggcaccgacttcaccctgaccatcagcagcctgcagcccgaggacttcgccacctactactgccagcagcactacaccaccccccccaccttcggccagggcaccaaggtggagatcaagaggaccgtggccgctcccagcgtgttcatcttcccccccagcgacgagcagctgaagtccggcaccgcctccgtggtgtgcctgctgaacaacttctacccccgcgaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgctga

SEQ ID NO: 69 - protein, 213 aa, Homo sapiens
MVPEEEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

(19) United States
(12) Patent Application Publication
(10) Pub. No.: US 2019/0330369 A1
(43) Pub. Date: Oct. 31, 2019

(54) COMBINATION OF ANTI-CD303 AND ANTI-HER2 ANTIBODIES

(71) Applicant: LABORATOIRE FRANÇAIS DU FRACTIONNEMENT ET DES BIOTECHNOLOGIES, LES ULIS (FR)

(72) Inventors: Abdessatar Sami CHTOUROU, ELANCOURT (FR); Nathalie FOURNIER, Erquinghem-Lys (FR); Christophe DE ROMEUF, LAMBERSART (FR)

(21) Appl. No.: 16/470,032

(22) PCT Filed: Dec. 15, 2017
(86) PCT No.: PCT/EP2017/083157

§ 371 Date: Jun. 14, 2019

(30) Foreign Application Priority Data
Dec. 16, 2016 (FR) 16 62603

(51) Int. Cl. C07K 16/32; C07K 16/28; A61P 35/00

(52) U.S. Cl. CPC C07K 16/32; C07K 16/2851; A61P 35/00; C07K 2317/20; A61K 2039/507; C07K 2317/24; C07K 2317/565; C07K 2317/41; C07K 2317/12; C07K 2317/21

(57) ABSTRACT

Disclosed is a pharmaceutical composition including, in a pharmaceutically acceptable carrier: a) at least one anti-CD303 antibody; and b) at least one anti-HER2 antibody. Also disclosed is the combination of the two aforementioned antibodies a) and b) to form combination products to be used simultaneously, separately or spread out over a period of time, for the prevention or treatment of HER2-positive cancer.

The subject of the present invention is a novel combination of antibodies, in particular for the prevention or treatment of HER2-positive cancer.

Breast cancer is the leading cause of death in women between 35 and 65 years of age. The most common breast cancers (95%) are adenocarcinomas which develop from the epithelial cells of the mammary gland. A distinction is made between cancers in situ and infiltrating cancers.

The staging of breast cancer is a major factor for prognosis. An early stage generates fewer risks of re-onset of the cancer (recurrence) and more favourable prognosis. A later stage generates a higher risk of recurrence and less favourable prognosis. The system the most frequently used to determine the stage of breast cancer is the TNM classification (Tumour, Nodes and Metastases). The TNM classification takes into account:
the size of the primary tumour;
the number of regional lymph nodes containing cancer cells, and where they are sited; and
propagation of the cancer, or metastases, towards another region of the body.

The most important stage-related factors are lymph node involvement and tumour size.

The size of a breast tumour increases the risk of recurrence:
a large breast tumour (5 cm or larger) generates the largest risk of recurrence;
a breast tumour having a size smaller than 1 cm and which has not propagated to the lymph nodes gives the best prognosis.

Involvement of the lymph nodes is also a major prognosis factor in breast cancer and independently of tumour size. It is assessed on the presence or non-presence of cancer in said nodes and the number of nodes involved. If the lymph nodes are positive, this indicates a higher risk of recurrence and less favourable prognosis than a breast cancer that has not propagated to the lymph nodes (negative nodes). In addition, breast cancer can also propagate to the internal mammary nodes without reaching the axillary nodes. In this case, this risk of recurrence is high even if the axillary nodes are negative. Finally, the higher the number of positive nodes the higher the risk of recurrence.

The Union for International Cancer Control (UICC) uses the TNM system to appraise the extent of breast cancer, and defines the following stages: stage 0, stage I, stages II A and B, stages IIIA to C and stage IV.

Once detected, different types of treatment can be used to treat breast cancer: surgery, radiotherapy, hormonotherapy, chemotherapy and targeted therapies. Individualisation of treatments according to patients is needed for optimal efficacy. It sometimes occurs that a single type of treatment is used. In other cases, an association of treatments is given to best control the disease. For example, treatment via surgery can be followed by treatment solely with chemotherapy or solely with radiotherapy. Several targeted therapies are currently used to combat breast cancer. These therapies, based on monoclonal antibodies or chemical molecules, block the mechanisms specific to cancer cells.

However, there still remains a need for an effective treatment against HER2-positive cancer, breast cancer in particular, especially when the cancer exhibits invasion of effector cells able to induce tolerance by the immune system, whilst identifying a therapy that is better tolerated and has fewer side effects.

The inventors have now surprisingly discovered that the combination of an anti-CD303 antibody and anti-HER2 antibody is particularly effective against HER2-positive tumours, in particular against breast tumours. The combination of these two antibodies can notably have a synergic effect at the site of the tumour.

The present invention therefore concerns a pharmaceutical composition comprising, in a pharmaceutically acceptable carrier:
a. at least one anti-CD303 antibody; and
b. at least one anti-HER2 antibody.

The present invention also relates to products containing:
a. at least one anti-CD303 antibody; and
b. at least one anti-HER2 antibody,

as combination products for simultaneous, separate or sequential use, for use in the prevention or treatment of HER2-positive cancer.

The invention also concerns:
at least one anti-HER2 antibody for use thereof in the prevention or treatment of HER2-positive cancer in combination with at least one anti-CD303 antibody; and
at least one anti-CD303 antibody for use thereof in the prevention or treatment of HER2-positive cancer, in combination with at least one anti-HER2 antibody.

The invention further concerns a method for treating HER2-positive cancer, comprising the administration to a patient of at least one anti-HER2 antibody and at least one anti-CD303 antibody.

In the present invention, the term «antibody» refers both to an immunoglobulin and to fragments and derivatives of this immunoglobulin. Immunoglobulins are well known to persons skilled in the art and are composed of an assembly of two dimers each formed of a heavy chain and a light chain. The multimeric complex is assembled by linking a light chain and a heavy chain via a disulfide bridge between two cysteines, the two heavy chains themselves also being linked together via two disulfide bridges.

Each of the heavy chains and light chains is composed of a constant region and a variable region. More specifically, each light chain is composed of a variable region (V_{L}) and a constant region (C_{L}). Each heavy chain is composed of a variable region (V_{H}) and a constant region formed of three constant domains C_{H1}, C_{H2} and C_{H3}. The domains C_{H2} and C_{H3} form domain Fc. The variable regions of the light chain and of the heavy chain are composed of three regions determining recognition of the antigen (CDR regions i.e. Complementary Determining Regions) surrounded by four framework regions (FR regions).

The anti-CD303 a) and anti-AMHRII b) antibodies can be monoclonal or polyclonal. Preferably, they are monoclonal antibodies.

The antibodies can be of several isotypes, as a function of their type of constant region: the constant regions e γ, α, μ, ε and δ respectively correspond to immunoglobulins IgG, IgA, IgM, IgE and IgD. Advantageously, the anti-CD303 a) and anti-HER2 b) antibodies are of IgG isotype. This isotype displays capability of inducing ADCC activity («Antibody-Dependent Cellular Cytotoxicity) in the largest number of (human) individuals. The γ constant regions comprise several subtypes: γ1, γ2, γ3, these three types of constant regions having the particularity of fixing human complement, and γ4, thereby creating the sub-isotypes IgG1, IgG2, IgG3 and IgG4. Advantageously, the anti-CD303 a) and anti-HER2 b) antibodies are of IgG1 or IgG2 isotype, preferably IgG1.

In one particular aspect of the invention, the anti-CD303 a) and anti-HER2 b) antibodies are selected from among murine antibodies, chimeric antibodies, humanized antibodies and human antibodies.

Preferably, the anti-CD303 a) antibody and/or anti-HER2 b) antibody is a chimeric antibody, and more preferably a chimeric antibody selected from among a murine/human chimeric antibody or human/macaque chimeric antibody.

Alternatively, and preferably, the anti-CD303 a) antibody and/or anti-HER2 b) antibody is a humanized antibody, and in particular a chimeric antibody in which the constant region of the heavy and light chains is of human origin.

The term «chimeric antibody» refers to an isolated antibody, in which its constituent sequence of each light chain and/or each heavy chain comprises or is composed of a hybrid sequence derived from at least two different animals. In particular, a chimeric antibody contains a light chain variable region and heavy chain variable region of murine wild-type, respectively fused with the light chain and heavy chain constant human regions. A chimeric antibody can be prepared using genetic recombination techniques well known to skilled persons.

The term «humanized antibody» refers to an antibody derived from an animal other than man and in which the sequences of the heavy chains and light chains other than CDRs have been replaced by corresponding sequences of one or more antibodies of human origin. The antibody is therefore mostly composed of human sequences, but the specificity thereof for the antigen imparted by the CDRs is derived from another species. In addition, some of the residues of backbone segments (called FRs) can be modified to maintain binding affinity (Jones et al. 1986; Verhoeyen et al. 1988; Riechmann et al. 1988). The humanized antibodies of the invention can be prepared using techniques known to skilled persons such as technologies of «CDR grafting», «resurfacing», SuperHumanisation, «Human string content», «FR libraries», «Guided selection», «FR shuffling» and «Humaneering», as summarized in the paper by Almagro et al. 2008.

As indicated above, the anti-CD303 and/or anti-HER2 antibody of the invention can also be present in the form of a fragment of anti-CD303 antibody and/or fragment of anti-HER2 antibody, respectively.

The term «fragment» refers in particular to the Fab, F(ab)′2 or Fd fragments.

The term «Fab» refers to an antibody fragment with a molecular weight of about 50 000 Da and having binding activity to the antigen. The Fab fragment is formed of the whole light chain (VL+CL) and part of the heavy chain (VH+CH1). It can be obtained in particular by treating IgGs with a protease, papain.

The term «F(ab′)2» refers to a fragment of about 100 000 Da and having binding activity to the antigen. It corresponds to the association via a disulfide bridge (hinge region) of two Fab fragments described above. It can be obtained by treating IgGs with a protease, pepsin.

The term «Fd» corresponds to that part of the heavy chain included in the Fab fragment. The Fd fragment is therefore formed of the domains VH and CH1.

As indicated above, the anti-CD303 and/or anti-HER2 antibody of the invention can also be present in the form of a derivative of an anti-CD303 a) antibody and/or derivative of an anti-HER2 b) antibody, respectively.

The term «derivative» particularly refers to scFv derivatives, and to multimers of scFv e.g. the diabody, triabody or tetrabody.

The term «scFv» (single chain Fv) is a VH:VL polypeptide synthesised using the genes encoding the VL and VH domains and a linker sequence. A scFv includes the CDRs maintained in a suitable conformation e.g. using genetic recombination techniques.

scFvs can also be used as basic modules for the development of multimer structures (dimeric: «diabody»; trimeric: «triabody»; tetrameric: «tetrabody»).

The term «diabody» refers to a dimer of scFv. This dimeric fragment has the property of maintaining the dual valence of the parent antibody. The diabody is bivalent, mono- or bi-specific depending on whether it fixes two same or different antigens.

The term «triabody» refers to the trivalent association of scFvs. A triabody can therefore fix three same or different antigens.

The term «tetrabody» refers to the tetravalent association of scFvs. A tetrabody is able to fix four same or different antigens.

The term «HER2-positive cancer» refers to a cancer comprising cells having the HER2 protein on their surface. In particular, said cancer cells overexpress the HER2 protein. For example, HER2-positive cancer is uterine cancer, ovarian cancer, breast cancer or stomach cancer. Preferably, the HER2-positive cancer is breast cancer. The term «breast cancer» refers to cancer of the mammary nodes, whether non-invasive (Ductal carcinoma In Situ DCIS) or invasive.

The present invention also concerns a pharmaceutical composition comprising, in a pharmaceutically acceptable carrier, at least one anti-CD303 antibody, and at least one fragment or derivative of anti-HER2 antibody.

The present invention also relates to products containing at least one anti-CD303 antibody, and at least one fragment or derivative of anti-HER2 antibody, as combination products for simultaneous, separate or sequential use, for use in the prevention or treatment of HER2-positive cancer.

The present invention also concerns a pharmaceutical composition comprising, in a pharmaceutically acceptable carrier, at least one fragment of anti-CD303 antibody, and at least one anti-HER2 antibody or one of the fragments thereof or one of the derivatives thereof.

The present invention also relates to products containing at least one fragment of anti-CD303 antibody, and at least one anti-HER2 antibody or one of the fragments thereof or one of the derivatives thereof, as combination product for simultaneous, separate or sequential use, for use in the prevention or treatment of HER2-positive cancer.

The present invention also concerns a pharmaceutical composition comprising, in a pharmaceutically acceptable carrier, at least one derivative of anti-CD303 antibody, and at least one anti-HER2 antibody or one of the fragments thereof or one of the derivatives thereof.

The present invention also relates to products containing at least one derivative of anti-CD303 antibody, and at least one anti-HER2 antibody or one of the fragments thereof or one of the derivatives thereof, as combination products for simultaneous, separate or sequential use, for use in the prevention or treatment of HER2-positive cancer.

The present invention also concerns a pharmaceutical composition comprising, in a pharmaceutically acceptable carrier, at least one anti-HER2 antibody, and at least one fragment or derivative of anti-CD303 antibody.

The present invention also relates to products containing at least one anti-HER2 antibody, and at least one fragment or derivative of anti-CD303 antibody, as combination products for simultaneous, separate or sequential use, for use in the prevention or treatment of HER2-positive cancer.

The present invention also concerns a pharmaceutical composition comprising, in a pharmaceutically acceptable carrier, at least one fragment of anti-HER2 antibody, and at least one anti-CD303 antibody or one of the fragments thereof or one of the derivatives thereof,

The present invention also relates to products containing at least one fragment of anti-HER2 antibody, and at least one anti-CD303 antibody or one of the fragments thereof or one of the derivatives thereof, as combination products for simultaneous, separate or sequential use, for use in the prevention or treatment of HER2-positive cancer.

The present invention also concerns a pharmaceutical composition comprising, in a pharmaceutically acceptable carrier, at least one derivative of anti-HER2 antibody, and at least one anti-CD303 antibody or one of the fragments thereof or one of the derivatives thereof.

The present invention also relates to products containing at least one derivative of anti-HER2 antibody, and at least one anti-CD303 antibody or one of the fragments thereof or one of the derivatives thereof, as combination products for simultaneous, separate or sequential use, for use in the prevention or treatment of HER2-positive cancer.

The present invention also concerns a pharmaceutical composition comprising, in a pharmaceutically acceptable carrier:
a. at least one anti-CD303 antibody; and
b. at least one anti-HER2 antibody.

By «pharmaceutically acceptable carrier» is meant a non-toxic medium compatible with a biological system such as a cell, cell culture, tissue or organism.

The invention also relates to products each containing one of the two above-mentioned active agents a) and b), said active agents being combined for simultaneous, separate or sequential use, and in the prevention or treatment of HER2-positive cancer.

Anti-CD303 Antibody

The anti-CD303 antibody is an antibody directed against the CD303 protein. This protein, also initially called BDCA-2, is specifically expressed on the surface of plasmacytoid dendritic cells, and it is a type II protein belonging to type-C lectins.

The human CD303 antigen (or CD303 protein) is the C member of lectin domain family 4 of type C (CLEC4 or «C-type lectin domain family 4, member C»). It is a type II transmembrane glycoprotein having 213 amino acids (accessible in Uniprot: Q8WTT0), comprising a short cytoplasmic domain without any evident signalling unit (amino acids 1-21), a transmembrane region (amino acids 22-44), and an extracellular domain (amino acids 45-213).

Plasmacytoid dendritic cells correspond to a sub-population of dendritic cells, also called DC2. Plasmacytoid dendritic cells are characterized by the Lin− markers (CD3−, CD19−, CD20−, CD14−, CD56−), HLA-DR+, CD11c−, CD123+ and CD45RA+). These cells have also been phenotypically characterized: they express the markers CD4, CD303 and BDCA-4. They are present in lymphoid organs and also circulate in the blood. They are capable of secreting type-I IFN in the presence of a viral infection. They can promote the growth of tumour cells and survival thereof, in particular by inducing an immunosuppressive environment in the environment of the tumour e.g. by inducing differentiation of regulator T lymphocytes T (Treg). These cells therefore have immunosuppressive and/or tolerogenic properties towards a tumour. The expression «immunosuppressive properties» refers to the properties of dendritic cells to develop and maintain immunosuppression in the tumoral environment. The expression «tolerogenic properties» means that plasmacytoid dendritic cells will not induce an immune response.

The use of anti-CD303 antibodies of the invention, through their cytotoxic action, advantageously allows suppression of the plasmacytoid dendritic cells infiltrating the tumour. The immunosuppressive and/or tolerogenic properties towards the tumour are therefore reduced, advantageously removed, thereby improving anti-tumour immunity in situ.

Preferably the anti-CD303 antibody of the invention is a monoclonal antibody directed against the ectodomain of the human CD303 antigen (SEQ ID NO: 69).

Advantageously, the anti-CD303 antibody of the invention comprises heavy chains comprising three CDR-Hs (heavy chain CDRs according to IMGT nomenclature) having the following amino acid sequences, or sequences having at least 80% identity with the following sequences, and light chains comprising three CDR-Ls (light chain CDRs according to IMGT nomenclature) having the following amino acid sequences or sequences having at least 80% identity with the following sequences:

i) CDR1-H-family 1: SEQ ID NO: 1; CDR2-H-family 1: SEQ ID NO: 2; CDR3-H-family 1: SEQ ID NO: 3; CDR1-L-family 1: SEQ ID NO: 4; CDR2-L-family 1: SEQ ID NO: 5; CDR3-L-family 1: SEQ ID NO: 6; or

ii) CDR1-H-family 2: SEQ ID NO: 7; CDR2-H-family 2: SEQ ID NO: 8, CDR3-H-family 2: SEQ ID NO: 9; CDR1-L-family 2: SEQ ID NO: 10; CDR2-L-family 2: SEQ ID NO: 11; CDR3-L-family 2: SEQ ID NO: 12.

The expression «at least 80% identity» means identity of 80%, 81%, 82%, 83%, 84%, 85%, 86%, 87%, 88%, 89%, 90%, 91%, 92%, 93%, 94%, 95%, 96%, 97%, 98%, 99% and 100%. The identity percentages to which reference is made in the present invention are determined on the basis of global alignment of the sequences to be compared i.e. on alignment of the sequences taken as a whole over their entire length using any algorithm known to skilled persons such as the Needleman-Wunsch algorithm—1970. This comparison of sequences can be carried out using any software known to skilled persons e.g. Needle software using a «Gap open» parameter of 10.0, «Gap extend» parameter of 0.5 and «Blosum 62» matrix. Needle software is available for example on the website ebi.ac.uk worldwide under the name «Align».

When the CDR or the variable region of an antibody has an amino acid sequence having at least 80%, preferably a least 85%, at least 90%, at least 91%, at least 92%, at least 93%, at least 94%, at least 95%, at least 96%, at least 97%, at least 98%, or at least 99% identity with a reference sequence, it may have insertions, deletions or substitutions compared with the reference sequence. If it has substitutions, substitution is preferably performed with an «equivalent» amino acid, i.e. any amino acid having a structure close to that of the original amino acid and is therefore not likely to modify the biological activities of the antibody, or an amino acid having a different structure but having intrinsic properties known to be equivalent to those of the original amino acid and not modifying the biological activities of the antibody.

Table 1 below summarises the amino acid sequences of the CDRs-IMGT of the two families of anti-CD303 antibodies able to be used in the invention:

TABLE 1
Amino acid sequences of the CDRs of the two
families of anti-CD303 antibodies able to be
used in the invention according to IMGT
nomenclature. In each sequence, X can represent
any amino acid.

| | Family 1 | Family 2 |
|---|---|---|
| CDR1-H | GYTFTDYS | GYTFTDXS |
| | (SEQ ID NO: 1) | (SEQ ID NO: 7) |
| CDR2-H | ISXYYGDX | INTETGXP |
| | (SEQ ID NO: 2) | (SEQ ID NO: 8) |
| CDR3-H | ARNXXXYXXXY | XRNGYYVGYYAXDY |
| | (SEQ ID NO: 3) | (SEQ ID NO: 9) |
| CDR1-L | QDIXNY | SSVXY |
| | (SEQ ID NO: 4) | (SEQ ID NO: 10) |
| CDR2-L | YTS | STS |
| | (SEQ ID NO: 5) | (SEQ ID NO: 11) |
| CDR3-L | QQGXTLPWT | QQRRSYPXT |
| | (SEQ ID NO: 6) | (SEQ ID NO: 12) |

Advantageously, the anti-CD303 antibody of the invention comprises three CDR-Hs (heavy chain CDRs according to IMGT nomenclature) having the following amino acid sequences, or sequences having at least 80% identity with the following sequences, and three CDR-Ls (light chain CDRs according to IMGT nomenclature) having the following amino acid sequences, or sequences having at least 80% identity with the following sequences:
i) CDR1-H-122A2: SEQ ID NO: 13, CDR2-H-122A2: SEQ ID NO: 14, CDR3-H-122A2: SEQ ID NO: 15, CDR1-L-122A2: SEQ ID NO: 16, CDR2-L-122A2: SEQ ID NO: 17, CDR3-L-122A2: SEQ ID NO: 18;
ii) CDR1-H-102E9: SEQ ID NO: 19, CDR2-H-102E9: SEQ ID NO: 20, CDR3-H-102E9: SEQ ID NO: 21, CDR1-L-102E9: SEQ ID NO: 22, CDR2-L-102E9: SEQ ID NO: 23, CDR3-L-102E9: SEQ ID NO: 24;
iii) CDR1-H-104C12: SEQ ID NO: 25, CDR2-H-104C12: SEQ ID NO: 26, CDR3-H-104C12: SEQ ID NO: 27, CDR1-L-104C12: SEQ ID NO: 28, CDR2-L-104C12: SEQ ID NO: 29, CDR3-L-104C12: SEQ ID NO: 30;
iv) CDR1-H-114D11: SEQ ID NO: 31, CDR2-H-114D11: SEQ ID NO: 32, CDR3-H-114D11: SEQ ID NO: 33, CDR1-L-114D11: SEQ ID NO: 34, CDR2-L-114D11: SEQ ID NO: 35, CDR3-L-114D11: SEQ ID NO: 36; or
v) CDR1-H-104E10: SEQ ID NO: 37, CDR2-H-104E10: SEQ ID NO: 38, CDR3-H-104E10: SEQ ID NO: 39, CDR1-L-104E10: SEQ ID NO: 40, CDR2-L-104E10: SEQ ID NO: 41, CDR3-L-104E10: SEQ ID NO: 42.

Advantageously, the anti-CD303 antibody of the invention has heavy and light chains with variable regions having the following amino acid sequences or sequences having at least 80% identity with the following sequences:
i) 122A2 antibody: heavy chain: SEQ ID NO: 43, light chain: SEQ ID NO: 48,
ii) 102E9 antibody: heavy chain: SEQ ID NO: 44, light chain: SEQ ID NO: 49,
iii) 104C12 antibody: heavy chain: SEQ ID NO: 45, light chain: SEQ ID NO: 50,
iv) 114D11 antibody: heavy chain: SEQ ID NO: 46, light chain: SEQ ID NO: 51, or
v) 104E10 antibody: heavy chain: SEQ ID NO: 47, light chain: SEQ ID NO: 52.

Table 2 below summarises the amino acid sequences of the CDRs and of the variable regions of the heavy and light chains of the anti-CD303 antibodies of the invention:

TABLE 2
Amino acid sequences of CDR1s, CDR2s, and CDR3s
of the heavy and light chains according to IMGT
nomenclature, and of the VH and VL fragments
of the anti-CD303 antibodies of the invention.
122A2 antibody
Heavy chain

| | |
|---|---|
| CDR1-H-IMGT-122A2 | GYTFTDYS (SEQ ID NO: 13) |
| CDR2-H-IMGT-122A2 | ISTYYGDS (SEQ ID NO: 14) |
| CDR3-H-IMGT-122A2 | ARNGNFYVMDY (SEQ ID NO: 15) |
| VH-122A2 | QVQLQQSGAELVRPGVSVKISCKGSGYTFTDYSMHWVK |
| | QSHAKSLEWIGVISTYYGDSNYNQKFKGKATMTVDKSST |
| | TAYMELARLTSEDSAIYYCARNGNFYVMDYWGQGTSVTV |
| | SS (SEQ ID NO: 43) |

Light chain

| | |
|---|---|
| CDR1-L-IMGT-122A2 | QDISNY (SEQ ID NO: 16) |
| CDR2-L-IMGT-122A2 | YTS (SEQ ID NO: 17) |
| CDR3-L-IMGT-122A2 | QQGNTLPWT (SEQ ID NO: 18) |
| VL-122A2 | DIQMTQTTSSLSASLGDRVTISCRASQDISNYLNWYQQKP |
| | DGTVKLLIYYTSRLHSGVPSRFSGSGSGTDYSLTISNLDQ |
| | EDIATYFCQQGNTLPWTFGGGTKLEIK |
| | (SEQ ID NO: 48) |

102E9 antibody
Heavy chain

| | |
|---|---|
| CDR1-H-IMGT-102E9 | GYTFTDYS (SEQ ID NO: 19) |
| CDR2-H-IMGT-102E9 | INTETGEP (SEQ ID NO: 20) |
| CDR3-H-IMGT-102E9 | TRNGYYVGYYAMDY (SEQ ID NO: 21) |
| VH-102E9 | QIHLVQSGPDLKKPGETVKISCKASGYTFTDYSMHWVKQ |
| | APGKGLKWMGWINTETGEPTYADDFKGRFAFSLESSAST |
| | AFLQINNLKNEDTSTYFCTRNGYYVGYYAMDYWGQGTSV |
| | TVSS (SEQ ID NO: 44) |

Light chain

| | |
|---|---|
| CDR1-L-IMGT-102E9 | SSVIY (SEQ ID NO: 22) |
| CDR2-L-IMGT-102E9 | STS (SEQ ID NO: 23) |
| CDR3-L-IMGT-102E9 | QQRRSYPFT (SEQ ID NO: 24) |
| VL-102E9 | QIVLTQSPAIMSASPGEKVTITCSASSSVIYIHWFQQKPGT |
| | SPKLWIYSTSYLASGVPARFSGSGSGTSYSLTISRMEAED |
| | AATYYCQQRRSYPFTFGGGTKLEIK (SEQ ID NO : 49) |

104C12 antibody
Heavy chain

| | |
|---|---|
| CDR1-H-IMGT-104C12 | GYTFTDYS (SEQ ID NO: 25) |
| CDR2-H-IMGT-104C12 | ISPYYGDT (SEQ ID NO: 26) |
| CDR3-H-IMGT-104C12 | ARNDDYYRFAY (SEQ ID NO: 27) |
| VH-104C12 | QVQLQQSGAELVGPGVSVKISCKGSGYTFTDYSMHWVK |
| | QSHAKSLEWIGVISPYYGDTNYNQKFKGKATMTVDKSSS |
| | TAYMELASLTSEDSAIYFCARNDDYYRFAYWGQGTLVTV |
| | SA (SEQ ID NO: 45) |

Light chain

| | |
|---|---|
| CDR1-L-IMGT-104C12 | QDINNY (SEQ ID NO: 28) |
| CDR2-L-IMGT-104C12 | YTS (SEQ ID NO: 29) |
| CDR3-L-IMGT-104C12 | QQGKTLPWT (SEQ ID NO: 30) |
| VL-104C12 | DLQMTQTPSSLSASLGDRVTISCRASQDINNYLSWYQEK |
| | PDGTFKLLIYYTSRLHSGVPSRFSGSGSGTDYSLTVRNLE |
| | QEDIGTYFCQQGKTLPWTFGGGTKLEIR |
| | (SEQ ID NO: 50) |

114D11 antibody
Heavy chain

| | |
|---|---|
| CDR1-H-IMGT-114D11 | GYTFTDSS (SEQ ID NO: 31) |
| CDR2-H-IMGT-114D11 | INTETGGP (SEQ ID NO: 32) |
| CDR3-H-IMGT-114D11 | ARNGYYVGYYALDY (SEQ ID NO: 33) |
| VH-114D11 | QIQLVQSGPELKKPGETVKISCKASGYTFTDSSMHWVQQ |
| | APNKGLKWMGWINTETGGPTYADDFKGRFAFSLETSART |
| | AYLQINNLKNEDTATYFCARNGYYVGYYALDYWGQGTSV |
| | TVSS (SEQ ID NO: 46) |

Light chain

| | |
|---|---|
| CDR1-L-IMGT-114D11 | SSVFY (SEQ ID NO: 34) |
| CDR2-L-IMGT-114D11 | STS (SEQ ID NO: 35) |
| CDR3-L-IMGT-114D11 | QQRRSYPYT (SEQ ID NO: 36) |
| VL-114D11 | QIVLTQSPAIMSASPGEKVTITCSASSSVFYMHWFQQKPG |
| | TSPKLWIYSTSNLASGVPARFSGSGSGTSYSLTISRMEAE |
| | DAATYYCQQRRSYPYTFGGGTKLEIK |
| | (SEQ ID NO: 51) |

104E10 antibody
Heavy chain

| | |
|---|---|
| CDR1-H-IMGT-104E10 | GYTFTDYS (SEQ ID NO: 37) |
| CDR2-H-IMGT-104E10 | INTETGEP (SEQ ID NO: 38) |
| CDR3-H-IMGT-104E10 | ARNGYYVGYYAMDY (SEQ ID NO: 39) |
| VH-104E10 | QIQLVQSGPELKKPGETVKISCKASGYTFTDYSMHWVKQ |
| | APGKGLKWMGWINTETGEPTYADDFKGRFAFSLETSATT |
| | AYLQINNFKNEDTATYFCARNGYYVGYYAMDYWGQGTS |
| | VTVSS (SEQ ID NO: 47) |

Light chain

| | |
|---|---|
| CDR1-L-IMGT-104E10 | SSVIY (SEQ ID NO: 40) |
| CDR2-L-IMGT-104E10 | STS (SEQ ID NO: 41) |
| CDR3-L-IMGT-104E10 | QQRRSYPYT (SEQ ID NO: 42) |
| VL-104E10 | QIVLTQSPAIMSASPGEKVTMTCSASSSVIYMHWFQQKP |
| | GTSPKLWIYSTSNLASGVPARFSGSGSGTSYSLTISRMEA |
| | EDAATYYCQQRRSYPYTFGGGTKLEIK |
| | (SEQ ID NO: 52) |

Preferably, the anti-CD303 antibody of the invention has a human constant region, preferably a human constant region of IgG1 isotype.

The preferred constant region sequences of the human heavy or light chains, SEQ ID NO: 53 and SEQ ID NO: 54, of IgG1 isotype, are given in Table 3 below.

TABLE 3
Preferred sequences of human heavy
and light chain constant regions
SEQ ID NO: 53 and SEQ ID NO: 54.

| | |
|---|---|
| Preferred | ASTKGPSVFPLAPSSKSTSGGTAALGCLVKDY |
| human heavy | FPEPVTVSWNSGALTSGVHTFPAVLQSSGLYS |
| chain constant | LSSVVTVPSSSLGTQTYICNVNHKPSNTKVDK |
| region (IgG1) | KVEPKSCDKTHTCPPCPAPELLGGPSVFLFPP |
| | KPKDTLMISRTPEVTCVVVDVSHEDPEVKFNW |
| | YVDGVEVHNAKTKPREEQYNSTYRVVSVLTVL |
| | HQDWLNGKEYKCKVSNKALPAPIEKTISKAKG |
| | QPREPQVYTLPPSRDELTKNQVSLTCLVKGFY |
| | PSDIAVEWESNGQPENNYKTTPPVLDSDGSFF |
| | LYSKLTVDKSRWQQGNVFSCSVMHEALHNHYT |
| | QKSLSLSPG (SEQ ID NO: 53) |
| Preferred | RTVAAPSVFIFPPSDEQLKSGTASVVCLLNNF |
| human light | YPREAKVQWKVDNALQSGNSQESVTEQDSKDS |
| chain constant | TYSLSSTLTLSKADYEKHKVYACEVTHQGLSS |
| region (IgG1) | PVTKSFNRGEC (SEQ ID NO: 54) |

Therefore, the anti-CD303 antibody of the invention advantageously comprises the heavy and light chains described in Table 4 below.

TABLE 4
Amino acid sequences of the heavy
and light chains of the anti-CD303
antibodies of the invention.

| Antibody | Heavy chain | Light chain |
|---|---|---|
| 122A2 | Fusion | Fusion |
| | SEQ ID NO: 43- | SEQ ID NO: 48- |
| | SEQ ID NO: 53 | SEQ ID NO: 54 |
| | (SEQ ID NO: 55) | (SEQ ID NO: 60) |
| 102E9 | Fusion | Fusion |
| | SEQ ID NO: 44- | SEQ ID NO: 49- |
| | SEQ ID NO: 53 | SEQ ID NO: 54 |
| | (SEQ ID NO: 56) | (SEQ ID NO: 61) |
| 104C12 | Fusion | Fusion |
| | SEQ ID NO: 45- | SEQ ID NO: 50- |
| | SEQ ID NO: 53 | SEQ ID NO: 54 |
| | (SEQ ID NO: 57) | (SEQ ID NO: 62) |
| 114D11 | Fusion | Fusion |
| | SEQ ID NO: 46- | SEQ ID NO: 51- |
| | SEQ ID NO: 53 | SEQ ID NO: 54 |
| | (SEQ ID NO: 58) | (SEQ ID NO: 63) |
| 104E10 | Fusion | Fusion |
| | SEQ ID NO: 47- | SEQ ID NO: 52- |
| | SEQ ID NO: 53 | SEQ ID NO: 54 |
| | (SEQ ID NO: 59) | (SEQ ID NO: 64) |

Anti-HER2 Antibody

The anti-HER2 antibody is an antibody directed against the Human Epidermal Growth Factor Receptor 2.

HER-2, also known as HER2/neu or ErbB2, is a member of the family of epidermal growth factor receptors. HER2 is a receptor tyrosine kinase bound to the plasma membrane and able to dimerize with itself and with other members of the family of epidermal growth factor receptors (HER1, HER2, HER3 et HER4). Dimerization, in turn, leads to activation of various intracellular biological pathways. HER2 is an oncogene overexpressed in a variety of cancers, including breast, ovarian, stomach and uterine cancers. Overexpression of HER2 in cancer (HER2-positive cancer or «HER2+ cancer») is associated with poor prognosis.

In particular, HER2 is the target of trastuzumab (Herceptin®), a monoclonal antibody which binds domain IV of the extracellular segment of the HER2/neu receptor. Trastuzumab received FDA approval in 1998 and has been used for the treatment of HER2+ breast cancer and HER2+ gastric cancer.

The anti-HER2 antibody of the invention can therefore be trastuzumab. However, trastuzumab, in its classical version, is not therapeutically effective in a large number of patients suffering from HER2+ cancers. Therefore, preferably, the anti-HER2 antibody of the invention is highly galactosylated trastuzumab, optionally highly fucosylated.

The heavy chain of trastuzumab has the following
sequence SEQ ID NO: 65:
(SEQ ID NO: 65)
MEFGLSWLFLVAILKGVQCEVQLVESGGGLVQPGGSLRLSCAASGFNI
KDTYIHWVRQAPGKGLEWVARIYPTNGYTRYADSVKGRFTISADTSKN
TAYLQMNSLRAEDTAVYYCSRWGGDGFYAMDYWGQGTLVTVSSASTKG
PSVFPLAPSSKSTSGGTAALGCLVKDYFPEPVTVSWNSGALTSGVHTF
PAVLQSSGLYSLSSWTVPSSSLGTQTYICNVNHKPSNTKVDKKVEPKS
CDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQV
SLTCLVKGFYPSDIAVEWESNGQPENNYKTTPPVLDSDGSFFLYSKLT
VDKSRWQQGNVFSCSVMHEALHNHYTQKSLSLSPGK
And the light chain has the following sequence
SEQ ID NO: 66:
(SEQ ID NO: 66)
MDMRVPAQLLGLLLLWLRGARCDIQMTQSPSSLSASVGDRVTITCRAS
QDVNTAVAWYQQKPGKAPKLLIYSASFLYSGVPSRFSGSRSGTDFTLT
ISSLQPEDFATYYCQQHYTTPPTFGQGTKVEIKRTVAAPSVFIFPPSD
EQLKSGTASVVCLLNNFYPREAKVQWKVDNALQSGNSQESVTEQDSKD
STYSLSSTLTLSKADYEKHKVYACEVTHQGLSSPVTKSFNRGEC

Preferably the anti-HER2 antibody is present in the form of a composition of monoclonal anti-HER2 antibodies, preferably in the form of a composition of trastuzumab, said composition comprising glycan structures at the Fc glycosylation sites (Asn 297 as per EU numbering) having a galactose content higher than 60%. EU numbering is generally used to designate a residue in a heavy chain constant region of an immunoglobulin (see EU numbering reported in Kabat et al., Sequences of Proteins of Immunological Interest, 5e Ed., Public Health Service, National Institutes of Health, Bethesda, Md. (1991)). The typical position of the glycosylated residue in an antibody is asparagine at position 297 («Asn297») according to EU numbering. It is to be noted that one of the monoclonal anti-HER2 antibodies described herein can be fully or partly purified.

The anti-HER2 antibodies can be glycosylated with a N-glycan at the Asn297 site of the heavy chain of the Fc region. Since antibodies generally comprise two heavy chains, they have up to two N-glycans in the Fc region. A variety of oligosaccharide profiles has been observed at this level, and in general the oligosaccharides found at this site include galactose, N-acetylglucosamine (GlcNAc), mannose, sialic acid, N-acetylneuraminic acid (NeuAc or NANA), N-glycolylneuraminic acid (NGNA) and fucose. The N-glycans found at Asn297 generally have a common basic structure composed of a non-branched chain of a first N-acetylglucosamine (GlcNAc), attached to the asparagine of the antibody, a second GlcNAc attached to the first GlcNAc and a first mannose attached to the second GlcNAc. Two additional mannoses are attached to the first mannose of the GlcNAc-GlcNAc-mannose chain, to complete the structure of the core (or pentasaccharide core) and provide two “arms” for the additional glycosylation. Also, the fucose residues can be attached to the first GlcNAc bound to Asn297.

The basic structure of the two arms is also known as an “antenna”. The most common type of N-glycan units of the antibodies present in the plasma is of complex type, namely composed of more than one type of monosaccharide. In the biosynthesis pathway for this N-glycan unit, several GlcNAc transferases attach the GlcNAc residues to the mannoses of the glycan core which can be lengthened with galactose, sialic acid and fucose residues. This glycosylation motif is called a «complex» structure.

A second glycosylation motif found on the «arms» of the basic N-glycan structure is a «high mannose» motif, characterized by additional mannoses (joined in branched form or in non-branched form).

A third glycosylation motif is a hybrid structure in which one of the arms is replaced by mannose whilst the other arm is complex.

A “galactosylated” anti-HER2 antibody such as used herein refers to any anti-HER2 antibody having at least one galactose monosaccharide in one of the N-glycans thereof. Galactosylated antibodies comprise antibodies in which both N-glycans each have motifs of complex type on each of the arms of the N-glycan units, antibodies in which both N-glycans have a motif of complex type on only one arm of the N-glycan units, antibodies which have only one N-glycan with motifs of complex type on each of the arms of the N-glycan, and antibodies which have only one N-glycan having a motif of complex type on only one of the arms of the N-glycan units. Galactosylated antibodies which comprise at least one galactose monosaccharide include the forms G1 (one galactose), G1F (one galactose, one fucose), G2 (two galactoses) and G2F (two galactoses, one fucose). In addition, the N-glycan which comprises at least one galactose monosaccharide can be sialylated or non-sialylated. It is to be noted also that N-glycans can also contain additional galactose residues, such as alpha-Gal, in one or more arms of the complex glycan unit, potentially leading to a N-glycan with four galactose groups.

A «highly galactosylated» anti-HER2 antibody, such as used herein, refers to an anti-HER2 antibody which comprises at least two galactose monosaccharides in the N-glycan units. Highly galactosylated antibodies comprise antibodies in which both N-glycans each have motifs of complex type on each of the arms of the N-glycan units, antibodies in which both N-glycans have a motif of complex type on only one arm of the N-glycan units, and antibodies which have one N-glycan with a motif of complex type on each of the arms of the N-glycan. Therefore, the highly galactosylated anti-HER2 antibodies of the invention correspond to the forms wherein the N-glycans each comprise a galactose in the glycan motif (e.g. G1 or G1F), wherein at least one N-glycan comprises two deux galactoses in the glycan motif (e.g. G2 or G2F) and wherein 3 or 4 galactoses are included in the glycan motif (e.g. (i) one N-glycan with a G1 glycan motif and one N-glycan with a G2 or G2F glycan motif, or (ii) two N-glycans with G2 or G2F). More preferably, the highly galactosylated anti-HER2 antibody comprises at least three galactose monosaccharides in the glycan motifs. In some embodiments, the highly galactosylated anti-HER2 antibody comprises at least four galactose monosaccharides in the glycan motifs.

The composition of highly galactosylated anti-HER2 antibodies of the invention preferably comprises a population of anti-HER2 antibodies (preferably trastuzumab) in which the degree of galactosylation of the antibodies in the population is at least 60%, preferably at least 70%. Preferably, the composition of highly galactosylated anti-HER2 antibodies of the invention comprises a population of anti-HER2 antibodies (preferably trastuzumab) in which the degree of fucosylation is also at least 50%, preferably at least 60%.

Preferably, the composition of highly galactosylated, optionally fucosylated, anti-HER2 antibodies, of the invention is a composition of trastuzumab produced in epithelial mammary cells of a non-human mammal, preferably a transgenic non-human mammal. Typically, the non-human mammal is selected from among goat, sheep, camel, cow, pig, rabbit, buffalo, horse, rat, mouse and llama. More particularly, the trastuzumab can be produced by transgenic goats, more specifically produced in the milk of transgenic goats.

An example of said transgenic goats is obtained as follows: trastuzumab-producing goats are generated using conventional micro-injection techniques (see for example U.S. Pat. No. 7,928,064). The cDNAs coding for the heavy and light chains (SEQ ID NO: 67 and SEQ ID NO: 68) are ligated with the expression vector of beta-casein to give the constructions BC2601 HC and LC BC2602. In these plasmids, the nucleic acid sequence encoding trastuzumab is under the control of a promoter facilitating the expression of trastuzumab in the mammary node of goats. The prokaryote sequences are removed and the DNA is micro-injected into goat pre-implantation embryos. These embryos are then transferred to pseudopregnant females. The resulting offspring is screened for the presence of transgenes. Offspring which carry both chains are identified as being transgenic founders. At a suitable age, the founders are bred. After pregnancy and parturition, they are milked.

The highly galactosylated and highly fucosylated trastuzumab composition is purified from the milk. Advantageously, reference is made for the production of transgenic anti-HER2 antibodies to the description given in the patent application published under number WO2014/125377 («Highly galactosylated anti-HER2 antibodies and uses thereof»).

The anti-CD303 and anti-HER2 antibodies of the invention can each independently be produced in a host cell, a non-human transgenic animal or transgenic plant comprising at least one nucleic acid coding for said antibody, the fragments or derivatives thereof, or a vector containing said nucleic acid.

Preferably, the anti-CD303 antibody and/or anti-HER2 antibody of the invention are produced via transgenesis, in particular in a non-human transgenic animal or transgenic plant.

The host cell can be of prokaryote or eukaryote origin, and selected in particular from among bacterial cells, insect, plant, yeast or mammalian cells. The antibody of the invention can then be produced by culture of the host cell under suitable conditions. A host cell of the invention can notably be obtained by transforming a cell line with the expression vector(s) of the heavy and light chains of an antibody of the invention, and by separating the different cell clones obtained. The transformed cell line is preferably of eukaryote origin and can be selected in particular from among insect, plant, yeast or mammalian cells. Suitable cell lines for producing antibodies notably include the lines selected from among: SP2/0; YB2/0; IR983F; Namalwa human myeloma; PERC6; CHO lines in particular CHO-K-1, CHO-Lec10, CHO-Lec1, CHO-Lec13, CHO Pro-5, CHO dhfr-, or CHO line with deletion of the two alleles encoding the FUT8 gene and/or GMD gene; Wil-2; Jurkat; Vero; Molt-4; COS-7; 293-HEK; BHK; K6H6; NSO; SP2/0-Ag 14, P3×63Ag8.653, duck embryo cell line EB66® (Valneva); rat hepatoma lines H4-II-E (DSM ACC3129) and H4-II-Es (DSM ACC3130) (see WO2012/041768), NM-H9D8 (DSM ACC2806), NM-H9D8-E6 (DSM ACC 2807) and NM H9D8-E6Q12 (DSM ACC 2856) (see WO2008/028686).

A non-human transgenic animal of the invention can be obtained by direct injection of the gene(s) of interest into a fertilized egg (Gordon et al. 1980). A non-human transgenic animal can also be obtained by inserting the gene(s) of interest into an embryonic stem cell and preparing the animal by chimera aggregation or chimera injection method (see Manipulating the Mouse Embryo, A Laboratory Manual, Second edition, Cold Spring Harbor Laboratory Press (1994); Gene Targeting, A Practical Approach, IRL Press at Oxford University Press (1993)). A non-human transgenic animal can also be obtained using a cloning technique wherein a nucleus, in which the gene(s) of interest have been inserted, is transplanted into an enucleated egg (Ryan et al. 1997; Cibelli et al. 1998, WO00/26357). A non-human transgenic animal producing an antibody of interest can be prepared with the above methods. The antibody can then be accumulated in the transgenic animal and harvested, in particular from the milk or eggs of the animal. For the production of antibodies in the milk of non-human transgenic animals, preparation methods are notably described in WO90/04036, WO95/17085, WO01/26455, WO2004/050847, WO2005/033281, WO2007/048077. Methods for purifying proteins of interest from the milk are also known (see WO01/26455, WO2007/106078). The non-human transgenic animals of interest particularly include mouse, rabbit, rat, goat, bovines (cow in particular) and poultry (chicken in particular).

A transgenic plant of the invention can be selected from among any plant allowing the production of antibodies. Numerous antibodies have already been produced in transgenic plants and the technologies required for obtaining a transgenic plant expressing an antibody of interest and for recovering the antibody are well known to skilled persons (see Stoger et al. 2002, Fisher et al. 2003, Ma et al. 2003, Schillberg et al. 2005). It is also possible to influence the glycosylation obtained in the plants, to obtain glycosylation close to that of the natural human antibodies (xylose-free), but additionally with low fucosylation e.g. by means of small interfering RNAs (Forthal et al. 2010).

The anti-CD303 antibody and anti-HER2 antibody are present, according to the invention, either in a pharmaceutical composition or as combination products.

They can therefore be combined with pharmaceutically acceptable excipients, and optionally with extended-release matrixes such as biodegradable polymers.

The pharmaceutical composition or combination products can be administered via oral, sublingual, subcutaneous, intramuscular, intravenous, intra-arterial, intrathecal, intra-ocular, intra-cerebral, transdermal, pulmonary, local or rectal route. The antibodies can be then be administered in unit administration form in a mixture with conventional pharmaceutical carriers. Unit administration forms comprise forms via oral route such as tablets, capsules, powders, granules and oral solutions or suspensions, sublingual and buccal administration forms, aerosols, subcutaneous, transdermal, topical, intraperitoneal, intramuscular, intravenous, subcutaneous, intrathecal implants, administration forms via intranasal route and rectal administration forms.

Preferably, the pharmaceutical composition or combination product contains a pharmaceutically acceptable carrier for a formulation able to be injected. In particular, these may be isotonic, sterile formulas, saline solutions (with monosodium or disodium phosphate, sodium, potassium, calcium or magnesium chloride and similar, or mixtures of said salts), or freeze-dried compositions which, when sterilized water or physiological saline solution accordingly are added thereto, allow the preparation of injectable solutions.

Suitable pharmaceutical forms for injectable use comprise sterile aqueous solutions or dispersions, oily formulations including sesame oil, groundnut oil, and sterile powders for extemporaneous preparation of sterile injectable solutions or dispersions. In all cases, the form must be sterile and fluid insofar as it must be injected through a syringe. It must be stable under production and storage conditions, and must be protected against contaminating action by microorganisms such as bacteria and fungi.

The dispersions of the invention can be prepared in glycerol, liquid polyethylene glycols or mixtures thereof, or in oils. Under normal conditions of storage and use, these preparations contain a preserving agent to prevent the growth of micro-organisms.

The pharmaceutically acceptable carrier can be a solvent or dispersion medium e.g. containing water, ethanol, a polyol (e.g. glycerine, propylene glycol, polyethylene glycol, and similar), suitable mixtures thereof and/or vegetable oils. Suitable fluidity can be maintained for example through the use of a surfactant such as lecithin. Prevention of action by microorganisms can be obtained via various antibacterial and antifungal agents e.g. parabens, chlorobutanol, phenol, sorbic acid or thimerosal. In many cases, it will be preferably to include isotonic agents e.g. sugars or sodium chloride. Extended absorption of the injectable compositions can be obtained through the use of absorption-delaying agents e.g. aluminium monostearate or gelatine.

Sterile injectable solutions are prepared by incorporating the active substances in required amount in a suitable solvent with some of the other ingredients listed above, optionally followed by filter sterilization. As a general rule, the dispersions are prepared by incorporating the various active sterilized ingredients in a sterile carrier containing the basic dispersion medium and the other ingredients required from among those listed above. With regard to sterile powders, to prepare sterile injectable solutions, the preferred preparation methods are vacuum drying and freeze-drying. At the time of formulation, the solutions are to be administered in a manner compatible with the dosage formulation in a therapeutically effective amount. The formulations are easily administered in a variety of pharmaceutical forms, such as the injectable solutions described above, but drug release capsules and the like can also be used. For parenteral administration in an aqueous solution for example, the solution must be suitably buffered and the liquid diluent made isotonic with sufficient saline solution or glucose. These particular aqueous solutions are particularly suitable for intravenous, intramuscular, subcutaneous and intraperitoneal administration. In this respect, the sterile aqueous media able to be used are known to those skilled in the art. For example, one dose can be dissolved in 1 ml of isotonic NaCl solution and then added to 1000 ml of suitable liquid. or injected at the proposed site of infusion. Some variations in dosage will necessarily have to be made as a function of the state of health of the patient being treated.

The level of therapeutically effective dose specific for a particular patient will depend on a variety of factors, including the disorder being treated and the seriousness of the disease, the activity of the specific compound employed, the specific composition used, patient age, bodyweight, general state of health, gender and diet, the time of administration, route of administration, the excretion rate of the specific compound used, the length of treatment or medications used in parallel.

The invention will now be illustrated with the following examples.

### EXAMPLES

Advantageously, in the present invention the anti-CD303 antibody used is a chimeric or humanized antibody comprising the CDR 122A2 sequences. The anti-HER2 antibody preferably used below is a trastuzumab antibody produced in transgenic goat milk e.g. such as described in application WO2014/125377.

### Example 1: Impact of pDC Depletion on ADCC and Phagocytosis Activities of Trastuzumab (Herceptin®)

1.1—Principle of the Study

pDCs are responsible for the differentiation of regulatory T cells (Moseman E A, 2004; Martin-Gayo E, 2010) inter alia via ICOS/ICOSL interaction (Ito, T., 2007; Faget, J. 2012; Faget, J. 2013). The regulatory T cells thus differentiated exert immunosuppressive mechanisms on the functions of the other cells in the immune system, NK cells in particular, via cell/cell contact but also via the secretion of immuno-modulating cytokines such as IL-10, IL-35 and TGF-β (Liu, C 2016).

Through cascade effect, it is therefore possible to examine the impact of pDC depletion on the action of an anticancer agent specific to a tumour involving in situ activation of pDCs. In particular, the protective effect of anti-CD303 antibodies targeting pDCs can be shown in a correlative study on the reduction or removal of IL-10 and TGF-β cytokines in the tumour environment, and the impact thereof on the effector functions of an administered anticancer agent (anti-HER2 antibody such as Herceptin®) specific to the tumour. According to this model, the anti-CD303 antibodies depleting the pDCs lead to limiting the immunosuppressive effects of regulatory T cells, thereby limiting their secretion of IL-10 and TGF-β. This limiting of IL-10 and TGF-β secretion is correlated with better ADCC of the NK cells, validating the indirect stimulating effect of the anti-CD303 antibodies on the anticancer action of anti-HER2 antibodies.

1.2—Effect of IL-10 and TGF-β on ADCC Activity of Trastuzumab

To test the ADCC of trastuzumab in a tumoral context involving either activation of pDCs or depletion of pDCs, the following protocol was set up:

NK effector cells transfected with CD16 («NK-CD16») were placed in culture at 3×10⁵ cells/ml in a culture medium containing IL-2.

At D-1, the NK-CD16 cells were placed in a culture medium without IL-2, in the absence or presence of IL-10 (5, 50 or 120 ng/ml) and TGF-β (5, 50 or 120 ng/ml).

At D0, BT-474 cells (breast tumour cells) (35000 cells/well) expressing HER-2 were incubated in a flat bottom 96-well plate with said NK-CD16 cells, in an E/T ratio (Effector—NK/Target—BT-474) of 5:1 and 5000 ng/ml of anti-HER-2 antibody (trastuzumab). After an incubation time of 16 hours at 37° C., the supernatant was collected.

The negative control followed the same protocol wherein trastuzumab was replaced by a chimeric antibody inhibiting the anti-FVIII antibody, produced in YB2/0.

Lysis of the target cells induced by the anti-HER-2 antibodies was measured chromogenically by quantifying the intracellular lactate dehydrogenase enzyme (LDH) released into the supernatant by the lysed target cells (Roche Diagnostics—Cytotoxicity Detection Kit LDH)

Percent lysis was calculated with the following formula:

% lysis=[(ER−SR)/(100−SR)]−[(NC−SR)/(100−SR)]

Where ER and SR respectively represent experimental (ER) and spontaneous (SR) release of LDH, and NC represents the natural cytotoxicity of NK cells.

The results (% lysis) are expressed as a percentage, 100% being the value taken as reference obtained with the NK-CD16 cells in the presence of trastuzumab and in the absence of IL-10 and TGF-β (i.e. Trastuzumab alone).

ADCC results are detailed in following Table 1:

TABLE 1

| | | | Trastu- | | | | Negative | |
|---|---|---|---|---|---|---|---|---|
| | Trastu- | | zumab + IL- | | Negative | | control + IL- | |
| | zumab | | 10/TGF-β | | control | | 10/TGF-β | |
| % lysis | 21 | 32 | 12 | 22 | 0 | 0 | 0 | 0 |
| 100% | 100 | 100 | 57 | 69 | 0 | 0 | 0 | 0 |
| corresponds to | | | | | | | | |
| the reference: | | | | | | | | |
| Trastuzumab | | | | | | | | |
| alone | | | | | | | | |

| | | | | |
|---|---|---|---|---|
| Mean | 100 | 63 | 0 | 0 |

These results show that the ADCC activity of trastuzumab is inhibited in the presence of IL-10 and TGF-β, compared with the control without these cytokines.

Based on an arbitrary value of 100% for trastuzumab alone, the mean percentage of ADCC is 63% in the presence of IL-10 and TGF-β at a concentration of 5000 ng/ml of trastuzumab.

### Conclusion

Through cascade effect, comparison of percent lysis observable in the absence of IL-10 and/or TGF-β cytokines, with percent lysis observable in the presence of these same cytokines, allows evaluation of the impact of pDC depletion at the tumour site. It can thus be shown that anti-CD303 antibodies allow indirect potentializing of the effect of the anti-HER2 anticancer antibodies.

2—Effect of IL-10 and TGF-β on Phagocytosis Induced by Trastuzumab

The monocytes were differentiated into CD16+ microphages (M2 like) for 2 days in RPMI 1640+10% SVF+M-CSF 50 ng/ml for 48 h.

The SKBR3 cells and macrophages were labelled with PKH-67 (green fluorescence) and PKH-26 (red fluorescence), respectively.

The SKBR3 cells were opsonized with 10 μg/ml of Trastuzumab antibody or with a control antibody and then incubated with the macrophages (1.105 of each cell/well) in the absence or presence of different concentrations of IL-10 (5, 50 or 120 ng/ml) alone, of TGF-β (5, 50 or 120 ng/ml) alone, and IL-10+ TGF-β.

After incubation for 3 h at 37° C., the cells were placed in a counting chamber (Mallassez) and observed under a fluorescence microscope.

Percent phagocytosis was evaluated by counting the number of macrophages (at least 100 macrophages) containing SKBR3 cells.

### Conclusion

Through cascade effect, comparison of percent phagocytosis observable in the absence of IL-10 and/or TGF-β cytokines, with percent lysis observable in the presence of these same cytokines, allows evaluation of the impact of pDC depletion at the tumour site. It is thus possible to show that the anti-CD303 antibodies allow indirect potentializing of the effect of the anti-HER2 anticancer antibodies.

### Example 2—Effect of Anti-CD303 Antibody on Activation of Regulatory T Cells (Treg)

2.1—Role of Anti-CD303 on the Phenotype and Expansion of Tregs in PBMC

The mononuclear cells (PBMC) were isolated from a tube of blood taken on anti-coagulant. The PBMCs did not contain pDCs. The Treg cells were identified and subjected to phenotype characterization via flow cytometry on the basis of 3 markers: CD4, CD25 and Fox-P3.

Different quantities of anti-CD303 antibody (from 1 ng to 10 μg/ml) were added to the PBMCs in the presence of IL-2 (500 U/ml). The number of Tregs and their phenotype were monitored over time (1 to 4 days).

Under the same conditions, beads coated with anti-CD3/anti-CD28 to stimulate T proliferation were added in a Treg/beads ratio of 4:1 to verify Treg activation.

### Conclusion

It can therefore be shown that the anti-CD303 antibodies, in the absence of pDCs, do not have any direct impact on the expansion and immunosuppressive phenotype of regulatory T cells.

2.2—Role of Anti-CD303 on the Phenotype and Expansion of Purified Tregs in the Presence of pDCs

The Treg cells (CD4⁺, CD25⁺) were purified from PBMCs using a 2-step process: depletion of negative CD4 cells (positive cells for the CD8, CD14, CD15, CD16, CD19, CD36, CD56, CD123, TCRγ/δ and CD235a markers) followed by positive selection of CD25⁺ cells.

Purified pDCs or pDC lines e.g. obtained with the method described in Maeda T et al., Int J Hematol. 2005 February; 81(2):148-54 (such as the CAL-1 line or a new line generated by retransfection of CD303 in Cal-1 and selection of a line stably expressing more than 40 000 CD303 antigen sites per cell, preferably between 40 000 and 50 000) were added in a Treg/pDC ratio of 100, 10 and 1. Different quantities of anti-CD303 antibody (from 1 ng to 10 μg/ml) were added to the Treg/pDC mixture in the presence of IL-2 (500 U/ml). The number of Tregs and their phenotype were monitored over time (1 to 4 days). Under the same conditions, beads coated with anti-CD3/anti-CD28 to stimulate T proliferation were added in a Treg/beads ratio of 4:1 to verify Treg activation. A negative control in the absence of pDCs was prepared, to verify the direct impact (expected to be neutral) of anti-CD303 on Tregs.

### Conclusion

Observation over several days of the expansion and differentiation of purified Tregs in the presence of pDCs, after administration of anti-CD303 antibody, can show that administration of anti-CD303 is effective in reducing or suppressing the immunosuppressive properties of pDCs.

### Example 3: Depletion of Human pDCs Via an Anti-CD303 Antibody In Vivo in the Treatment of Breast Cancer

3.1-Generation of a Reproductive Study Model of a Pathological Situation in Man

To study the effect of an anti-CD303 antibody in the treatment of breast cancer, a humanized tumour mouse model (HTM) can be used. It is characterized by the development of a mature human immune system and the growth of human breast cancer cells previously co-transplanted with the human hemopoietic stem cells. Very recently, the effectiveness of treatment with Trastuzumab/Herceptin was proven in this model (Wege A K et al., Oncotarget 2017, 8(2): 2731-2744).

This model advantageously allows the grouping together of several relevant elements for reproducibility under in vivo conditions: presence of human pDCs which alone express the target CD303 on their surface, presence of infiltration by human Treg cells, presence of human tumour cells expressing HER2 on their surface, molecule targeted by trastuzumab/Herceptin®, and an immunocompetent murine host (effector cells of NK type for ADCC activity). The model previously described in the article by Wege et al. (Int. J. Cancer 2011: 129, 2194-2206) combines all these characteristics.

This model was adapted to make it compatible with BRGSF™ or BRGSF™-A2 mice (BALB/c, Rag2tm1Fwa, IL-2Rγctm1Cgn, SIRPαNOD, Flk2tm1Irl, Tg(HLA-A/H2-D/B2M)1Bpe) characterized by the absence of T, B, and NK mouse cells, and solely expressing the HLA of human class 1, HLA-A2.1. Said BRGSF™ mice can be generated following the procedure described by Legrand N, Huntington N D, Nagasawa M et al. (Functional CD47/signal regulatory protein alpha (SIRP(alpha)) interaction is required for optimal human T- and natural killer- (NK) cell homeostasis in vivo. Proc. Natl. Acad. Sci. U.S.A 2011; 108:13224-13229). They acquire the genotype (HLA-A/H2-D/B2M)^{1Bpe} via transgenesis (Pascolo, S., N. Bervas, J. M. Ure, A. G. Smith, F. A. Lemonnier, and B. Perarnau. 1997. HLA-A2.1-restricted education and cytolytic activity of CD8(+) T lymphocytes from beta2 microglobulin (beta2m) HLA-A2.1 monochain transgenic H-2Db beta2m double knockout mice. J Exp Med 185:2043-2051).

3.2—Method Able to be Used to Test the Activity of the Anti-CD303 Antibody

In brief, new-born mice derived from the line of immunodeficient BRGSF-A2 mice (BALB/c Rag2^{tm1Fwa} IL-2Rδ_{c}^{tm1Cgn} SIRPα^{NOD} Flk2^{tm1Irl} Tg(HLA-A/H2-D/B2M)^{1Bpe} are irradiated (3 Gy) over the first 192 hours of their lifetime. Twenty-four hours later they are transplanted by intra-hepatic injection with 1.5×10⁵ human CD34+ cells isolated from umbilical cord blood (CB) in the presence or absence of 3×10⁶ COV434-AMHRII-Luc tumour cells (expressing luciferase for bioluminescent tracking). BT474 cells are cells of human origin derived from breast carcinoma expressing the HER2/Neu receptor on their surface: the target of the Trastuzumab antibody. In this particular model, the BT474 cells, before administration thereof, were modified by lentiviral transduction to cause them to become luminescent through the constituent expression of luciferase. This modification provides transverse tracking of tumour penetration over time by means of bioluminescence analysis, whilst not sacrificing the animals and thereby best adjusting the window for start of treatment. Eleven to twelve weeks after co-administration of human cells, the mice were tested for their extent of humanization by analysing the cell composition of their blood (human and murine) by flow cytometry, and divided into five different groups (cf. Table 2 below): a control group without injection of BT474 cells, treated with an isotype antibody (i.e. an anti-Factor VIII inhibiting antibody) (this group was used as negative control for tumour penetration—Group 1); a control group with injection of BT474 cells, treated with an isotype antibody (i.e. an anti-Factor VIII inhibiting antibody) (Group 2); a group treated with transgenic trastuzumab (TTG)—Group 3); a group treated with the anti-CD303 antibody (Group 4); and a group given the combination of anti-CD303 antibody and TTG treatments (Group 5).

The doses administered and treatment frequencies are given in Table 2 below:

| Groups | BT474-luc⁺ | Tested product(s) | Number |
|---|---|---|---|
| 1 | No | Isotype | n = 10 |
| | | (anti-Factor VIII | |
| | | inhibiting antibody) | |
| 2 | Yes | Isotype | n = 10 |
| | | (anti-Factor VIII | |
| | | inhibiting antibody) | |
| 3 | Yes | TTG | n = 10 |
| 4 | Yes | Anti-CD303 | n = 10 |
| 5 | Yes | TTG + anti-CD303 | n = 10 |

Treatment started 14 weeks after humanization (i.e. injection of human CD34+ cells isolated from umbilical cord blood—CB) in the presence or absence of 3×10⁶ BT474-Luc tumour cells, and lasted 19 weeks.

For treatment, the tested products were injected via intravenous route at a dose of 30 mg/kg bodyweight for the anti-CD303 antibody every 3 days, and once a week at 10 mg/kg bodyweight for the TTG antibody. The bodyweight of the mice was determined 3 days before the start of treatment for individual dose adjustment.

Blood samples were frequently taken to test the efficacy of human pDC depletion, by flow cytometry. In addition, bioluminescent analysis at the start of treatment and subsequently every two weeks was performed to compare the efficacy of the different tested products. Finally, tumour analysis in three animals per group at week 18 after humanization was carried out by flow cytometry to verify the presence or absence of human pDCs infiltrating the tumours.

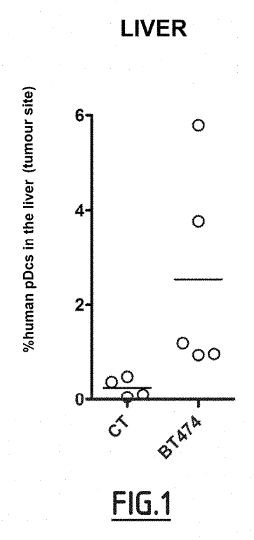

Results:
The impact of the treatment on the human pDC sub-population, and the other populations of lymphoid cells (B lymphocytes B, T lymphocytes . . . ) present in the blood and spleen, was determined by flow cytometry at different times: D1, D3 and D7. The results show that treatment with the anti-CD303 antibody at a dose of 30 mg/kg in humanized BRGSF-HIS mice induces depletion of human pDCs for at least 7 days in the blood and spleen. In the blood, the depleting activity on human pDCs was rapid (>80% on Day 1) but occurred later in the spleen. In the blood and spleen, depletion of human pDCs was always efficient (>90%) on Day 7, i.e. 3 days after the last injection of the monoclonal anti-CD303 antibody. It is to be noted that the depleting activity of the anti-CD303 antibody was highly specific since it did not significantly affect the other sub-populations of human hemopoietic cells in the tested organs.
Additionally, it is to be noted that this model proves to be a particularly suitable model for the study of the invention, since the detecting of pDCs in the tumours of sacrificed mice for the first time shows the presence of pDCs infiltrating these tumours (cf. FIG. 1 giving the percentage of human pDcs in the liver—tumour site—in the negative control (mice not injected with BT474 cells—(CT)), and in mice injected with BT474 cells (BT474)).

### Conclusion

The adapted HTM mouse model can advantageously be used to evaluate the indirect impact of anti-CD303 antibody administration on the effect of the anti-breast tumour agent: the anti-HER2 antibody (trastuzumab), under conditions reproducing a physiological situation in vivo, in particular by comparing the results obtained with the different tested groups.

This model is therefore useful in evaluating the benefit, and advantageously the synergic effect, of administering an anti-CD303 antibody in combination with administration of an anti-HER2 antibody in a breast tumour.

1-11. (canceled)

12. Pharmaceutical composition comprising, in a pharmaceutically acceptable carrier:
a. at least one anti-CD303 antibody; and
b. at least one anti-HER2 antibody.

13. A method for preventing or treating HER2-positive cancer in a subject in need thereof, comprising administering to said subject products containing:
c. at least one anti-CD303 antibody; and
d. at least one anti-HER2 antibody,
which are combination products for simultaneous, separate or sequential use.

14. The pharmaceutical composition according to claim 12, wherein the antibodies a) and b) are selected from among mouse antibodies, chimeric antibodies, humanized antibodies and human antibodies.

15. The pharmaceutical composition according to claim 12, wherein the anti-CD303 antibody is a monoclonal antibody directed against the ectodomain of the human CD303 antigen.

16. The pharmaceutical composition according to claim 12, wherein the anti-CD303 antibody comprises the following CDRs:

| | |
|---|---|
| CDR1-H-IMGT-122A2 | GYTFTDYS (SEQ ID NO: 13) |
| CDR2-H-IMGT-122A2 | ISTYYGDS (SEQ ID NO: 14) |
| CDR3-H-IMGT-122A2 | ARNGNFYVMDY (SEQ ID NO: 15) |
| CDR1-L-IMGT-122A2 | QDISNY (SEQ ID NO: 16) |
| CDR2-L-IMGT-122A2 | YTS (SEQ ID NO: 17) |
| CDR3-L-IMGT-122A2 | QQGNTLPWT (SEQ ID NO: 18) |
| or else: | |
| CDR1-H-IMGT-102E9 | GYTFTDYS (SEQ ID NO: 19) |
| CDR2-H-IMGT-102E9 | INTETGEP (SEQ ID NO: 20) |
| CDR3-H-IMGT-102E9 | TRNGYYVGYYAMDY (SEQ ID NO: 21) |
| CDR1-L-IMGT-102E9 | SSVIY (SEQ ID NO: 22) |
| CDR2-L-IMGT-102E9 | STS (SEQ ID NO: 23) |
| CDR3-L-IMGT-102E9 | QQRRSYPFT (SEQ ID NO: 24) |
| or else: | |
| CDR1-H-IMGT-104C12 | GYTFTDYS (SEQ ID NO: 25) |
| CDR2-H-IMGT-104C12 | ISPYYGDT (SEQ ID NO: 26) |
| CDR3-H-IMGT-104C12 | ARNDDYYRFAY (SEQ ID NO: 27) |
| CDR1-L-IMGT-104C12 | QDINNY (SEQ ID NO: 28) |
| CDR2-L-IMGT-104C12 | YTS (SEQ ID NO: 29) |
| CDR3-L-IMGT-104C12 | QQGKTLPWT (SEQ ID NO: 30) |
| or else: | |
| CDR1-H-IMGT-114D11 | GYTFTDSS (SEQ ID NO: 31) |
| CDR2-H-IMGT-114D11 | INTETGGP (SEQ ID NO: 32) |
| CDR3-H-IMGT-114D11 | ARNGYYVGYYALDY (SEQ ID NO: 33) |
| CDR1-L-IMGT-114D11 | SSVFY (SEQ ID NO: 34) |
| CDR2-L-IMGT-114D11 | STS (SEQ ID NO: 35) |
| CDR3-L-IMGT-114D11 | QQRRSYPYT (SEQ ID NO: 36) |
| or else: | |
| CDR1-H-IMGT-104E10 | GYTFTDYS (SEQ ID NO: 37) |
| CDR2-H-IMGT-104E10 | INTETGEP (SEQ ID NO: 38) |
| CDR3-H-IMGT-104E10 | ARNGYYVGYYAMDY (SEQ ID NO: 39) |
| CDR1-L-IMGT-104E10 | SSVIY (SEQ ID NO: 40) |
| CDR2-L-IMGT-104E10 | STS (SEQ ID NO: 41) |
| CDR3-L-IMGT-104E10 | QQRRSYPYT (SEQ ID NO: 42) |

17. The pharmaceutical composition according to claim 12, wherein the anti-HER2 antibody is a composition of trastuzumab, said composition comprising glycan structures at the Fc glycosylation sites having a galactose content higher than 60%.

18. The pharmaceutical composition according to claim 12, wherein the anti-HER2 antibody is a composition of trastuzumab, said composition comprising glycan structures at the Fc glycosylation sites having a degree of fucosylation of at least 50%.

19. The pharmaceutical composition according to claim 12, wherein the anti-HER2 antibody is trastuzumab produced in the milk of transgenic goats.

20. The pharmaceutical composition according to claim 12, wherein the anti-CD303 antibody and/or anti-HER2 antibody is selected from among Fab, F(ab)′2, Fd, scFv and multimers of scFv.

21. A method for preventing or treating HER2-positive cancer in a subject in need thereof, comprising administering to said subject a pharmaceutical composition according to claim 12, wherein the HER2-positive cancer is selected from among uterine, ovarian, breast and stomach cancer.

22. A method for preventing or treating HER2-breast positive cancer in a subject in need thereof, comprising administering to said subject a pharmaceutical composition according to claim 12.

23. The pharmaceutical composition according to claim 12, wherein the anti-HER2 antibody is a composition of trastuzumab, said composition comprising glycan structures at the Fc glycosylation sites having a galactose content higher than 70%.

24. The pharmaceutical composition according to claim 12, wherein the anti-HER2 antibody is a composition of trastuzumab, said composition comprising glycan structures at the Fc glycosylation sites having a degree of fucosylation of at least 60%.

25. The method according to claim 13, wherein the antibodies a) and b) are selected from among mouse antibodies, chimeric antibodies, humanized antibodies and human antibodies.

26. The method according to claim 13, wherein the anti-CD303 antibody is a monoclonal antibody directed against the ectodomain of the human CD303 antigen.

27. The method according to claim 13, wherein the anti-CD303 antibody comprises the following CDRs:

| | |
|---|---|
| CDR1-H-IMGT-122A2 | GYTFTDYS (SEQ ID NO: 13) |
| CDR2-H-IMGT-122A2 | ISTYYGDS (SEQ ID NO: 14) |
| CDR3-H-IMGT-122A2 | ARNGNFYVMDY (SEQ ID NO: 15) |
| CDR1-L-IMGT-122A2 | QDISNY (SEQ ID NO: 16) |
| CDR2-L-IMGT-122A2 | YTS (SEQ ID NO: 17) |
| CDR3-L-IMGT-122A2 | QQGNTLPWT (SEQ ID NO: 18) |
| or else: | |
| CDR1-H-IMGT-102E9 | GYTFTDYS (SEQ ID NO: 19) |
| CDR2-H-IMGT-102E9 | INTETGEP (SEQ ID NO: 20) |
| CDR3-H-IMGT-102E9 | TRNGYYVGYYAMDY (SEQ ID NO: 21) |
| CDR1-L-IMGT-102E9 | SSVIY (SEQ ID NO: 22) |
| CDR2-L-IMGT-102E9 | STS (SEQ ID NO: 23) |
| CDR3-L-IMGT-102E9 | QQRRSYPFT (SEQ ID NO: 24) |
| or else: | |
| CDR1-H-IMGT-104C12 | GYTFTDYS (SEQ ID NO: 25) |
| CDR2-H-IMGT-104C12 | ISPYYGDT (SEQ ID NO: 26) |
| CDR3-H-IMGT-104C12 | ARNDDYYRFAY (SEQ ID NO: 27) |
| CDR1-L-IMGT-104C12 | QDINNY (SEQ ID NO: 28) |
| CDR2-L-IMGT-104C12 | YTS (SEQ ID NO: 29) |
| CDR3-L-IMGT-104C12 | QQGKTLPWT (SEQ ID NO: 30) |
| or else: | |
| CDR1-H-IMGT-114D11 | GYTFTDSS (SEQ ID NO: 31) |
| CDR2-H-IMGT-114D11 | INTETGGP (SEQ ID NO: 32) |
| CDR3-H-IMGT-114D11 | ARNGYYVGYYALDY (SEQ ID NO: 33) |
| CDR1-L-IMGT-114D11 | SSVFY (SEQ ID NO: 34) |
| CDR2-L-IMGT-114D11 | STS (SEQ ID NO: 35) |
| CDR3-L-IMGT-114D11 | QQRRSYPYT (SEQ ID NO: 36) |
| or else: | |
| CDR1-H-IMGT-104E10 | GYTFTDYS (SEQ ID NO: 37) |
| CDR2-H-IMGT-104E10 | INTETGEP (SEQ ID NO: 38) |
| CDR3-H-IMGT-104E10 | ARNGYYVGYYAMDY (SEQ ID NO: 39) |
| CDR1-L-IMGT-104E10 | SSVIY (SEQ ID NO: 40) |
| CDR2-L-IMGT-104E10 | STS (SEQ ID NO: 41) |
| CDR3-L-IMGT-104E10 | QQRRSYPYT (SEQ ID NO: 42) |

28. The method according to claim 13, wherein the anti-HER2 antibody is a composition of trastuzumab, said composition comprising glycan structures at the Fc glycosylation sites having a galactose content higher than 60%.

29. The method according to claim 13, wherein the anti-HER2 antibody is a composition of trastuzumab, said composition comprising glycan structures at the Fc glycosylation sites having a degree of fucosylation of at least 50%.

30. The method according to claim 13, wherein the anti-HER2 antibody is trastuzumab produced in the milk of transgenic goats.

31. The method according to claim 13, wherein the anti-CD303 antibody and/or anti-HER2 antibody is selected from among Fab, F(ab)′2, Fd, scFv and multimers of scFv.
